(19) United States
(12) Patent Application Publication
(10) Pub. No.: US 2015/0216978 A1
(43) Pub. Date: Aug. 6, 2015

(54) MODULATORS OF DRUGS USING CARBOXYLESTERASE SELECTIVE INHIBITORS

(71) Applicant: Rhode Island Board of Education, State of Rhode Island and Providence Plantations, Providence, RI (US)

(72) Inventor: Bingfang Yan, Saunderstown, RI (US)

(21) Appl. No.: 14/561,694

(22) Filed: Dec. 5, 2014

Related U.S. Application Data
(60) Provisional application No. 61/912,257, filed on Dec. 5, 2013.

(51) Int. Cl. A61K 47/22; A61K 31/575

(52) U.S. Cl. CPC A61K 47/22; A61K 31/575

(57) ABSTRACT

A method is disclosed of using an inhibitor of a carboxylesterase and a drug in an animal, wherein the inhibited carboxylesterase reduced the efficacy of the drug in the animal.

### CROSS REFERENCE TO RELATED APPLICATION

This application claims priority from U.S. Provisional Application Ser. No. 61/912,257 filed Dec. 5, 2013, the entire content and substance of which is incorporated by reference herein in its entirety.

### SUPPORT

This invention was made with government support under Grant No. ES007965 awarded by National Institutes of Health. The government has certain rights in the invention.

### BACKGROUND

Drugs and cosmetics are foreign chemicals and human bodies have developed several systems to get rid of these chemicals. Carboxylesterases, a class of enzymes, constitute one of the systems. These enzymes hydrolyze drugs such as aspirin, irinotecan, plavix and tamiflu. Approximate 20% of drugs currently on the market undergo hydrolysis. Hydrolysis of drugs may have opposite therapeutic significance depending on a drug. In the case of anticancer agent irinotecan, for example, hydrolysis is required for its therapeutic activities. In contrast, hydrolysis of the heart medicines aspirin and plavix represents inactivation.

Carboxylesterases have long been recognized as targets for enhancing the efficacy and/or decreasing the toxicity of therapeutic agents metabolized by carboxylesterases. In other words, tremendous efforts have been made in both academic communities and pharmaceutical industries to develop and identify inhibitors of carboxylesterases. A difficulty in this endeavor, however, has been the existence of multiple carboxylesterases and poor selectivity, and further this has likely adverse safety affects as well. To the Applicant's knowledge, no carboxylesterase inhibitors are used clinically as of today.

Humans have several carboxylesterases, however, only carboxylesterase-1 (CES1) and carboxylesterase-2 (CES2) have been established to play critical roles in the metabolism of drugs and others materials including cosmetics. While both CES1 and CES2 catalyze hydrolysis, they exhibit profound differences in substrate specificity. For example, CES1 hydrolyzes tamiflu (anti-influenza virus) and plavix, whereas CES2 hydrolyzes aspirin and irinotecan. In addition, both CES1 and CES2 are abundant in the liver but only CES2 is abundant in the gastrointestinal tract, the kidney and the skin. The expression of CES2 in various organs was previously reported by the Applicant.

### SUMMARY OF THE INVENTION

In accord with the present invention, a potent and irreversible inhibitor of CES2, a carboxylesterase known to metabolize many drugs and cosmetics, is provided by the use of Orlistat, also known as tetrahydrolipstatin. It has been discovered that Orlistat can be used to enhance the efficacy or reduce toxicity of drugs and cosmetics that are metabolized by CES2. A further embodiment is that Orlistat is useful for other carboxylesterases when higher concentrations are used.

In accordance with another embodiment, the inhibitory activity of orlistat on CES2 was detected by pretreatment or together with a drug. Orlistat can be formulated with a drug or cosmetic to enhance the efficacy or reduce the toxicity. The formulation can be so designed that Orlistat is absorbed prior to the drug or the cosmetic. The formulation can also be so designed to increase the absorption.

In yet another embodiment, the inhibitory potency of Orlistat varies and is dependent upon on a carboxylesterase. It is well known that carboxylesterases show similar 3-D structures. The invention is that derivatives structurally of Orlistat are inhibitors of carboxylesterases, Orlistat can serve as a prototype for developing selective inhibitors of respective carboxylesterases.

### BRIEF DESCRIPTION OF THE DRAWINGS

The following description may be further understood with reference to the accompanying drawings in which:

FIGS. 1A and 1B show illustrative graphical representations of the relative sensitivity of CES2 (FIG. 1B) as compared with CES1 (FIG. 1A) to Orlistat inhibition, wherein Orlistat at 1 nM inhibited CES23 by 75% (FIG. 1B), but no inhibition was detected with CES1 (FIG. 1A);

FIG. 2 shows an illustrative microphotographic representation of dominant activity bands in liver samples due to inhibition of CES2 as compared with inhibition of CES1;

FIGS. 3A and 3B show illustrative microphotographic representations of activity staining (FIG. 3A) and immune-blotting (FIG. 3B) as a function of Orlistat inhibition of CES1 and CES2; and

FIGS. 4A and 4B show illustrative graphical representations of relative cell viability corresponding to treatment with Olistat and/or PPD, an anticancer producing drug, (FIG. 4A), and microphotographic representations of growth status of cells treated with DMSO, Orlistat, PPD or both PPD and Orlistat (FIG. 4B).

### DETAILED DESCRIPTION

Orlistat is known as a potent and irreversible inhibitor of carboxylesterases, particularly CES2. In the present invention, it was shown that Orlistat at 1 nM inhibited CES2 activity by 75%, placing CES2 as the most sensitive target of Orlistat. Further, it shown that pretreatment with Orlistat reduced the cell killing activity of PPD (an anticancer prodrug) by as much as 60%. The resulting invention showed for the first time that Orlistat can be used to enhance the efficacy or reduce toxicity of drugs and cosmetics that are metabolized by carboxylesterases, noticeably CES2. More preferably, Orlistat was able to modulate metabolic activities of drug compounds. The invention further allows optimization of more preferred formulations of a drug or a cosmetic using Orlistat to effect bioefficacy of the compound in vvivo. Most preferably, derivatives structurally similar to Orlistat are inhibitors of carboxylesterases and Orlistat can serve as a prototype for developing selective inhibitors of respective carboxylesterases.

This invention discovers that: (1) Orlistat can be used to enhance the efficacy or reduce toxicity of drugs and cosmetics that are metabolized by carboxylesterases, noticeably CES2; (2) such modulatory activities can be optimized by proper formulations of Orlistat with a drug or a cosmetic; (3) derivatives structurally similar to Orlistat are inhibitors of carboxylesterases and Orlistat can serve as a prototype for developing selective inhibitors of respective carboxylesterases.

### Example 1

### Differential Inhibition of Carboxylesterases

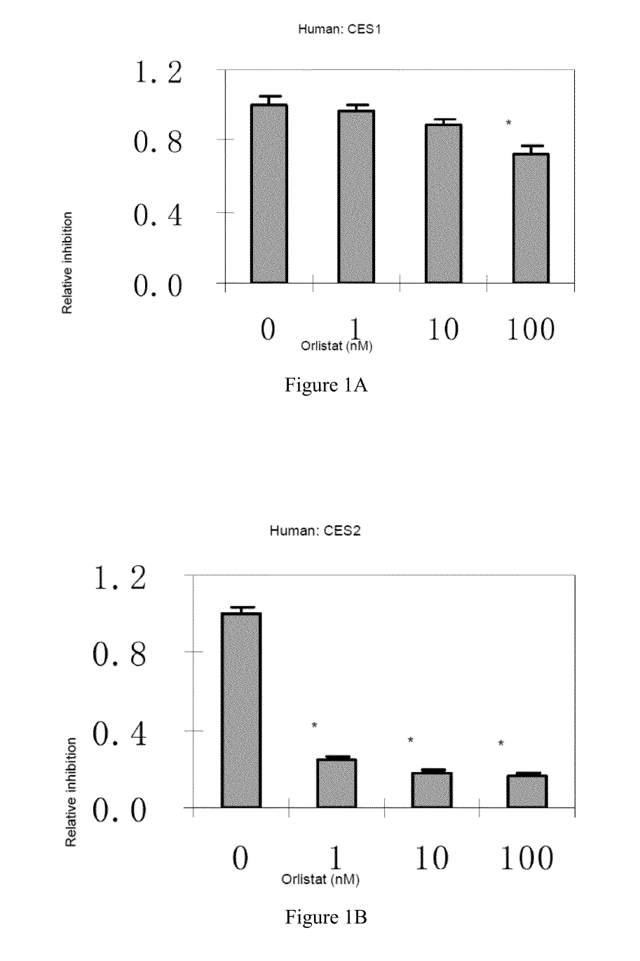

For a decade, Orlistat has been the most used anti-obesity drug. However, the use of Orlistat has been associated with fatal liver and kidney toxicity. This type of toxicity is rare, pointing to a compound effect of multiple factors. Specifically, we assumed that orlistat alters the activity of detoxification enzymes, which may have fatal effects when these factors are perfectly aligned. To shed light on this possibility, we tested Orlistat for the inhibition of carboxylesterases, a class of hydrolytic enzymes with known detoxication significance. As shown in FIGS. 1A and 1B, human CES2 but not CES1 (both are human enzymes) was highly sensitive to Orlistat inhibition. Orlistat at 1 nM inhibited CES2 by 75%, but no inhibition was detected with CES1. Even at 100 nM orlistat, CES1 was inhibited by ˜30% only (as shown in FIG. 1B). It should be noted that mouse ces2c and ces2e were potently inhibited by 1 nM Orlistat; namely 90 and 55%, respectively, in contrast, ces1d was inhibited by 12% only at this concentration (Biochem Pharmacol. 2013, 85:439-447). In a striking contrast, rat ces1d and ces1e, two major forms of liver CESs, were resistant to Orlistat (Biochem Pharmacol. 2013, 85:439-447).

### Example 2

### Irreversibility

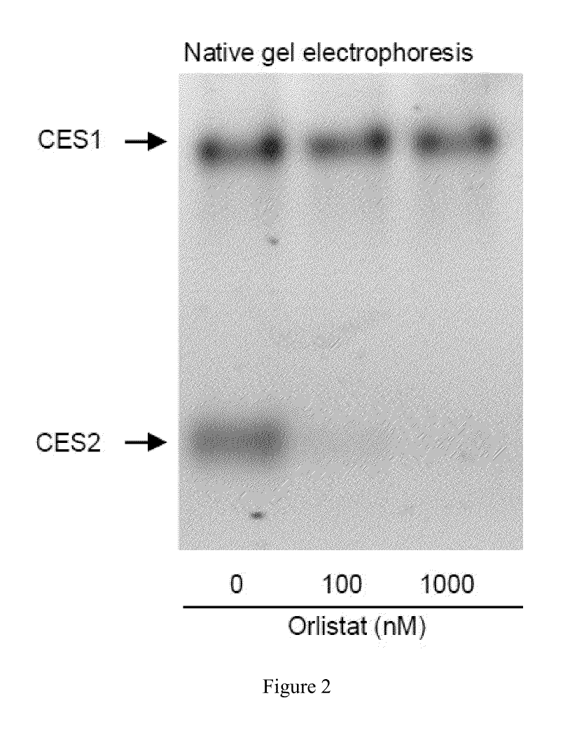

It was tested whether the inhibition involves covalent interactions between Orlistat and CES2. Microsomes from human livers (CES1 and CES2) were incubated with Orlistat, resolved by native gel electrophoresis to remove free Orlistat, and stained for the remaining activity. As shown in FIG. 2, two dominant activity bands were detected in human liver microsomes, corresponding to CES1 and CES2. Once again, the activity of CES2 but not CES1 was profoundly reduced upon incubation with Orlistat. It should be emphasized that the inhibition detected by this method with the removal of Orlistat by electrophoresis established that Orlistat is an irreversible inhibitor of CES2 (Biochem Pharmacol. 2013, 85:439-447).

### Example 3

### Cellular Inhibition

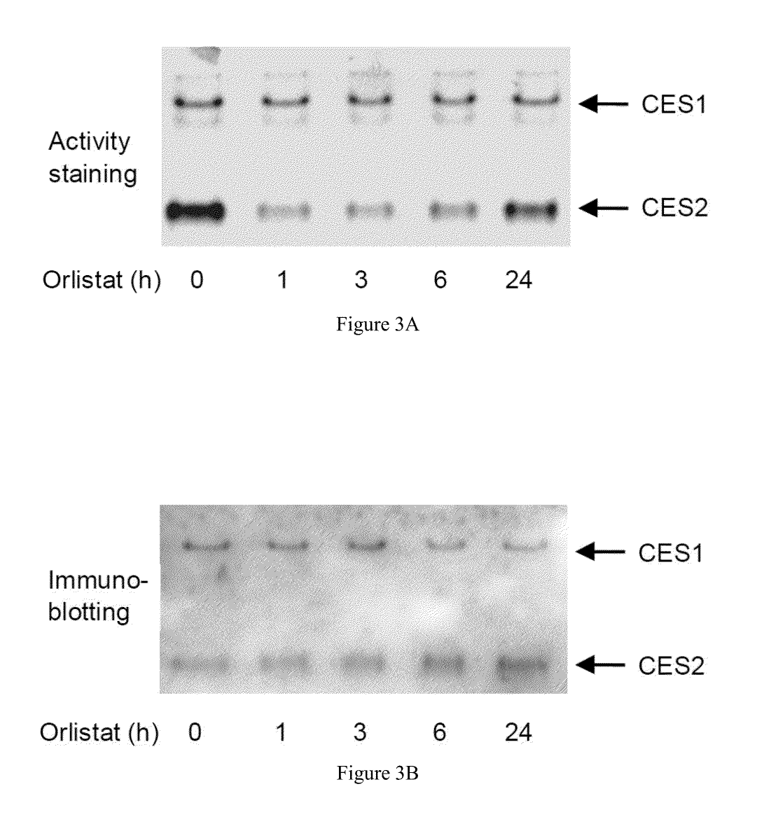

The cellular activity of Orlistat was examined on the inhibition of CES2 within cells. To test this possibility, LS180 cells (a colon adenocarcinoma line) were treated with orlistat for 1-24 h, washed extensively to remove free Orlistat and lysed by sonication. The lysates were then tested for the hydrolytic activity by native gel electrophoresis. As shown in FIGS. 3A and 3B, the activity of CES2 but not CES1 was inhibited by ˜80% within 1-h incubation (FIG. 3A). Comparable inhibition was detected when cells were treated for 6 h. Interestingly, prolonged inhibition (i.e., 24 h) was less effective. We next tested whether the less inhibition by prolonged incubation was due to increased expression of CES2. Western blotting was performed with the same gel stained for activity. As predicated, prolonged incubation increased CES2 expression (FIG. 3B). These findings established that regeneration of CES2 requires new synthesis of CES2.

### Example 4. Clinical Implication

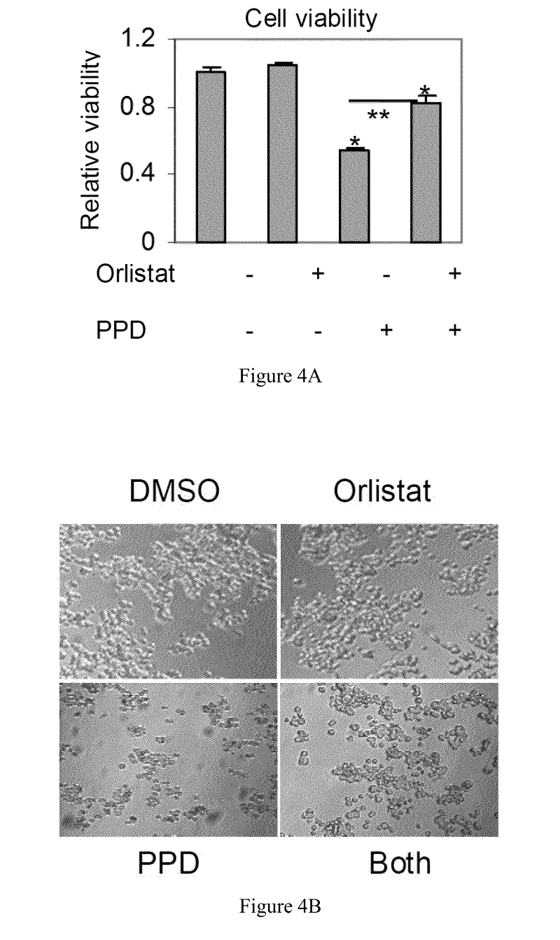

To shed light on the clinical consequences of inhibited CES2 by Orlistat, cells were treated with PPD in the presence or absence of Orlistat. PPD is a carbamate anticancer prodrug and activated preferentially by CES2 hydrolysis of the ester side chain of the carbamate (20, 21). As shown in FIGS. 4A and 4B, treatment with Orlistat alone caused no changes in cell viability, whereas treatment with PPD alone caused significant reduction in cell viability (FIG. 4A). The reduction of cell viability by carbamate was significantly reversed by Orlistat. FIG. 4B shows the representative image of growth status of cells treated with Orlistat, PPD or both (Biochem Pharmacol. 2013, 85:439-447). This finding was confirmed in CES2 induced condition (Brit J Pharmacol. 2013, 168:1989-1999).

what is claimed is:

1. A method of using an inhibitor of a carboxylesterase and a drug in an animal, wherein the inhibited carboxylesterase reduced the efficacy of the drug in the animal.

2. The method of claim 1, wherein the inhibition of the carboxylesterase is a preferred isozyme.

3. The method of claim 1, wherein the inhibition of the preferred isozyme is carboxylesterase 2 and not carboxylesterase 1.

4. A method of providing a drug that includes the steps of providing the drug together with tetrahydrolipstatin, and inhibiting carboxylesterase.

5. The method of claim 4, wherein the inhibition of the carboxylesterase is a preferred isozyme.

6. The method of claim 4, wherein the inhibition of the preferred isozyme is carboxylesterase 2 and not carboxylesterase 1.

7. A chemical compound that includes a drug together with tetrahydrolipstatin for improving the efficacy of the drug in a subject.

8. The chemical compound of claim 7, carboxylesterase is inhibited by the tetrahydrolipstatin.

9. The chemical compound of claim 8, wherein the inhibition of the carboxylesterase is a preferred isozyme.

10. The method of claim 8, wherein the inhibition of the preferred isozyme is carboxylesterase 2 and not carboxylesterase 1.
